(19) United States
(12) Patent Application Publication
(10) Pub. No.: US 2021/0253633 A1
(43) Pub. Date: Aug. 19, 2021

(54) SINGLE PASS TANGENTIAL FLOW FILTRATION HYBRID CONFIGURATIONS FOR ENHANCING CONCENTRATION OF MACROMOLECULE SOLUTIONS

(71) Applicant: BRISTOL-MYERS SQUIBB COMPANY, Princeton, NJ (US)

(72) Inventors: Abhiram Arunkumar, Watertown, MA (US); Nripen Singh, Acton, MA (US); Junyan Zhang, Cambridge, MA (US)

(21) Appl. No.: 16/973,422

(22) PCT Filed: Jun. 6, 2019
(86) PCT No.: PCT/US2019/035746

§ 371 Date: Dec. 8, 2020

Related U.S. Application Data
(60) Provisional application No. 62/682,326, filed on Jun. 8, 2018.

(51) Int. Cl. C07K 1/34; C12N 15/10; C12N 9/36; B01D 61/14

(52) U.S. Cl. CPC C07K 1/34; C12N 15/1017; C12N 9/2462; C12Y 302/01017; B01D 61/145; B01D 2319/06; B01D 2315/16; B01D 2315/10; B01D 2317/022; B01D 2319/022; B01D 61/142

(57) ABSTRACT

This disclosure provides a method for concentrating a solution of a macromolecule that is retained on at least two semi-permeable membranes that have different molecular weight cutoffs (MWCOs), the method comprising passing the solution through a hybrid configuration of said semi-permeable membranes staged in series in a single pass tangential flow filtration (SPTFF) apparatus. The method is applicable to the efficient concentration of biological macromolecules such as proteins, antibodies and nucleic acids.

### CROSS REFERENCE TO RELATED APPLICATION

This application claims the benefit of U.S. Provisional Application No. 62/682,326, filed Jun. 8, 2018, the content of which is hereby incorporated herein by reference in its entirety.

Throughout this application, various publications are referenced in parentheses by author name and date, by Patent No. or Patent Publication No., or by Internet website. Full citations for these publications may be found at the end of the specification immediately preceding the claims. The disclosures of these publications are hereby incorporated in their entireties by reference into this application in order to more fully describe the state of the art as known to those skilled therein as of the date of the invention described and claimed herein. However, these disclosures are expressly incorporated by reference into the present application only to the extent that no conflict exists between the incorporated information and the information provided by explicit disclosure herein. Moreover, the citation of a reference herein should not be construed as an acknowledgement that such reference is prior art to the present invention.

### FIELD OF THE INVENTION

This invention relates to a process for enhancing the concentration of a solution of a macromolecule using a hybrid configuration of semi-permeable ultrafiltration membranes that have different molecular weight cutoffs in single pass tangential flow filtration (SPTFF) at a high feed flow rate.

### BACKGROUND OF THE INVENTION

Significant progress has been made in biologics process development to increase upstream productivity (titers). As a result, the downstream purification platform is continuously evolving to increase capacity and selectivity to handle the increased biomass (Konstantinov and Cooney 2015). In addition to improved capacity, selectivity, better utilization of capacities and uniformity in product quality, one of the primary benefits of continuous processing is in the cost of the drug substance: it has been reported that manufacturing operating costs have been reduced five-fold (from $1230 per gram for a batch process to $250 per gram for a continuous process) with a three-fold decrease in capital costs (Hammerschmidt et al. 2014; Zydney 2015).

In order to enable continuous processing, continuous chromatography and multicolumn column chromatography need to form the workhorse of the purification process. There have been significant advances in continuous chromatography involving, for example, companies adapting a variant of a continuous bioprocessing method in their manufacturing pipeline (Warikoo et al. 2012). Whereas perfusion and technologies like alternating tangential flow filtration are widely used to manufacture therapeutic proteins and harvest them continuously, and chromatography is used to perform the purification to acceptable standards, the final step in producing the drug substance involves concentration of the protein and exchanging the protein into the formulation buffer. This unit operation is traditionally performed in the batch recirculation mode using an ultrafiltration membrane that is retentive to the protein but permeable to buffer components. The recirculation of the protein solution makes the operation a batch process. Batch tangential flow filtration (TFF) has been the subject of extensive research in order to produce highly concentrated monoclonal antibodies (mAbs) and Fc fusion proteins (Arunkumar et al. 2016; Baek et al. 2017; Binabaji et al. 2016). However, the adaptation of continuous processing methods in the final step to concentrate proteins and exchange them in the formulation buffer has been a slow process.

Single pass tangential flow filtration (SPTFF) is a technology that eliminates the recirculation loop and allows for concentration in a single pump pass. This is achieved by increasing the residence time of the protein solution within the module and increasing the effective length and area simultaneously. Past studies reported the use of commercially available SPTFF modules to concentrate proteins, and highlighted the key hydraulic differences between TFF and SPTFF (Dizon-Maspat et al. 2012). Much of the work on SPTFF has been on completely retained proteins using retentive membranes that had a molecular weight cut-off of 10 kDa or 30 kDa (Arunkumar et al. 2017; Nambiar et al. 2018; Brinkmann et al. 2018). There are no data available comparing the behavior of partially retained solutes and completely retained solutes using SPTFF, or the effect of membrane molecular weight cut-off on achieving concentrated protein solutions. Such data will be required before membrane steps can replace chromatographic polishing steps (Zydney 2016) or before SPTFF can be used to isolate the product of interest in the permeate. This is also true for emerging new modalities such as purifying viral vector, plasmid DNA, or RNA, when the product of interest appears in the permeate and SPTFF may be the best option to achieve purification objectives.

In the case of retained mAbs, SPTFF has been examined as an alternative to TFF to achieve highly concentrated solutions (Dizon-Maspat et al. 2012). Several studies have reported on the complex TFF behavior of concentrated mAb solutions and the dependence on the inter-molecular interactions in the protein and the buffer composition, and their interaction with the module hydraulics. For example, Binabaji et al. found that the type of the screen channel combined with the buffer affects the maximum achievable concentration (Binabaji et al. 2016; Baek et al. 2017), and this has been confirmed by Arunkumar et al. (2016).

This application describes the sieving behavior of biological macromolecules in SPTFF using ultrafiltration membranes of different molecular weight cutoffs (MWCOs), and on the effect of membrane MWCO and the screen type, exemplified by the sieving behavior of a partially retained protein, lysozyme (molecular mass=14.3 kDa) and two completely retained mAbs (molecular mass=140-150 kDa). A hybrid MWCO solution was identified as the ideal solution for use in obtain high concentrations at higher feed flow rates. The data disclosed herein show substantial differences between TFF and SPTFF for partially retained and completely retained proteins, and provides the basis for the methods described herein for separating proteins using SPTFF that are widely applicable in the biotechnology, biopharmaceutical and food processing industries, among others.

### SUMMARY OF THE INVENTION

The present invention provides a method for concentrating a solution of a macromolecule, such as a biological macromolecule, that is retained on at least two semi-permeable membranes that have different molecular weight cutoffs (MWCOs), the method comprising passing the solution through a hybrid configuration of said semi-permeable membranes staged in series in a SPTFF apparatus, wherein the final membrane in the series of membranes has a larger MWCO than the preceding membrane or membranes. In certain embodiments, the biological macromolecule is a protein, for example, a polypeptide, an antibody or a Fc fusion protein.

In certain embodiments, three semi-permeable membranes are used in the SPTFF apparatus. In certain preferred embodiments, the method is used to concentrate an antibody (molecular mass of about 140-150 kDa) and the membranes are staged in a 30-30-50 kDa hybrid configuration.

In certain embodiments, the method is used for concentrating an antibody solution and achieves a concentration factor, i.e., the fold increase in concentration over the starting solution, of at least 10. For example, a concentration factor of about 10, 12, 15, 20, 30, 50, 60, 70, 75, 90, 100 or 150-fold may be achieved. In further embodiments, the method achieves a concentration of antibody of up to about 150 to about 200 mg/mL.

In certain other embodiments, the present method for concentrating a biological macromolecule using membranes with two different MWCOs in series in a hybrid configuration allows operation at a higher maximum flow rate than that achieved using membranes in a non-hybrid configuration. In certain embodiments, the flow rate is at least 2-fold higher than in a non-hybrid configuration. In certain other embodiments, the flow rate is at least 4-fold higher.

Other features and advantages of the instant invention will be apparent from the following detailed description and examples which should not be construed as limiting. The contents of all cited references, including scientific articles, patents and patent applications cited throughout this application are expressly incorporated herein by reference.

### BRIEF DESCRIPTION OF THE FIGURES

FIG. 1 shows the flow path in SPTFF. The area of all the stages is shown as the same, but could be different in principle. The dashed lines indicate the filtration membrane. Although three stages, each containing one semi-permeable membrane are depicted, different numbers of stages could be employed. For example, depending on the objective, two (e.g., 20-30 or 30-50 hybrid configurations), four, five, or more stages could be employed.

FIG. 2 shows the variation in observed sieving coefficients of lysozyme with changes in filtrate flux using different membranes in the recirculation TFF mode at a normalized feed flow rate of 100 L/h/m². No concentration was performed, only total-recycle of retentate and permeate into the feed container.

FIG. 3 shows stage-wise sieving coefficients of lysozyme using different membranes in the SPTFF mode. Data is presented for each stage as a function of feed flow rate.

FIG. 4 shows: (A) Normalized feed flow rate versus final retentate concentration using different SPTFF arrangements for mAb1. The feed concentration was 16 g/L±3 g/L. (B) Differential pressure versus final retentate concentration for different SPTFF arrangements for mAb1.

FIG. 5 shows: (A) Normalized feed flow rate versus final retentate concentration using different SPTFF arrangements for mAb2. The feed concentration was 5 g/L±1 g/L. (B) Differential pressure versus final retentate concentration for different SPTFF arrangements for mAb2.

FIG. 6 shows the cumulative volume concentration factor at each stage for (A) mAb1 and (B) mAb2, using the 30 kDa, 50 kDa and the 30-30-50 kDa hybrid configurations. The 50 kDa membrane for mAb1 was not used beyond stage 2 for the 50-50-50 A configuration. Normalized feed flow rates were: (A) mAb1: 3 g/L feed solution 7.8 L/h/m² for the 30 kDa, 11.7 L/h/m² for the 50 kDa, and 12.1 L/h/m² for the 30-30-50 kDa hybrid; (B) 16 g/L feed solution: 7.5 L/h/m² for the 30 kDa, 6.6 L/h/m² for the 50 kDa, and 21.1 L/h/m² for the 30-30-50 kDa hybrid.

### DETAILED DESCRIPTION OF THE INVENTION

The present invention relates to efficient, high feed-flow-rate methods for concentrating a solution of a macromolecule, such as a biological macromolecule, using a hybrid configuration of semi-permeable membranes that have different MWCOs and are staged in series in a SPTFF apparatus.

The study described herein examined several important aspects of the ultrafiltration behavior of partially retained solutes and completely retained proteins using SPTFF. The resulting dataset is the first experimental data reported for the ultrafiltration behavior of a partially retained solute using SPTFF, and the first dataset for a completely retained protein (a mAb) using more open membranes and hybrid membrane configurations. As reported in Examples 2 and 3, the sieving behavior of partially retained solutes (exemplified with lysozyme) in SPTFF is complicated compared to conventional TFF. This complicated behavior will translate to other more complex systems where SPTFF is being explored, including for the isolation of mAbs in the permeate during primary clarification (Bolton et al. 2017) and for purification of other modalities where high sieving into the permeate is desired. While the operation itself is simpler than TFF, the sieving behavior is more complex. In the case of completely retained proteins, SPTFF is already in place in industry to provide inline concentration of in-process pools as reported in the literature (Arunkumar et al. 2017; Teske et al. 2010). One of the major limitations of SPTFF is the need to explore complicated staging arrangements or expand the membrane as higher concentrations are targeted because the normalized feed flow rates (<10 L/h/m²) are significantly lower. The present studies explored the use of a hybrid staging arrangement that used retentive membranes of two different MWCOs to achieve the target concentrations at higher flow rates than currently obtained using SPTFF.

### Terms

In order that the present disclosure may be more readily understood, certain terms are first defined. As used in this application, except as otherwise expressly provided herein, each of the following terms shall have the meaning set forth below. Additional definitions may be provided throughout the application.

An “antibody” (Ab) is a glycoprotein immunoglobulin (Ig) which binds specifically to an antigen and comprises at least two heavy chains and two light chains interconnected by disulfide bonds. Each heavy chain comprises a heavy chain variable region and a heavy chain constant region. The heavy chain constant region of an IgG Ab comprises three constant domains, whereas each light chain comprises a light chain variable region and a light chain constant region. The light chain constant region of an IgG Ab comprises one constant domain. The term “Fc region” or “Fc domain” refers to a C-terminal region of an Ig heavy chain that contains at least a portion of the constant region. The term “antibody” is used herein in the broadest sense and encompasses various antibody structures, including but not limited to monoclonal antibodies (mAbs), polyclonal Abs, and multispecific Abs (e.g., bispecific Abs). The term “monoclonal” Ab (mAb) refers to a non-naturally occurring preparation of Ab molecules of single molecular composition, i.e., Ab molecules whose primary sequences are essentially identical and which exhibit a single binding specificity and affinity for a particular epitope. An “antigen-binding portion” of an Ab (also called an “antigen-binding fragment”) refers to one or more fragments of an Ab that retain the ability to bind specifically to the antigen bound by the whole Ab.

An “Fc fusion protein” is a protein comprising an Ig Fc domain directly linked to another peptide. Frequently, the fused partner has therapeutic potential, and it is attached to the Fc domain to endow the fusion protein with additional beneficial biological and pharmacological properties, e.g., to increase the plasma half-life of the therapeutic protein, decrease renal clearance for larger sized molecules, or to enable interaction with Fc receptors found on immune cells, which is necessary for mediating antibody-dependent cell-mediated cytotoxicity (ADCC) and complement-dependent cytotoxicity (CDC).

The terms “feed,” “feed sample” and “feed stream” refer to the solution being fed to the filtration module for separation. The feed sample is typically separated by a filtration membrane into two streams, a permeate stream and a retentate stream.

A “filtration membrane” or “semi-permeable membrane” refers to a selectively permeable membrane for separating a feed into a permeate stream and a retentate stream using a TFF process. The terms “permeate stream” and “permeate” refer to the portion of the feed that has permeated through a filtration membrane. The terms “retentate stream” and “retentate” refer to the portion of the solution that has been retained by a filtration membrane. Depending on membrane porosity, filtration membranes include ultrafiltration, microfiltration, reverse osmosis and nanofiltration membranes. Microfiltration membranes, with pore sizes typically between about 0.1 μm and about 10 μm, are generally used for clarification, sterilization, and removal of microparticulates or for cell harvesting. Ultrafiltration membranes, with much smaller pore sizes between about 1 nm and about 100 nanometers, are used for concentrating and desalting dissolved molecules (e.g., proteins, peptides, nucleic acids, carbohydrates, and other biomolecules), exchanging buffers, and gross fractionation. Ultrafiltration membranes are typically classified by MWCO rather than pore size, and TFF typically utilizes ultrafiltration membranes ranging from about 1 to 1000 kDa MWCOs to retain different size molecules. In TFF processes, the filtration membrane is contained within a “cassette”, which is a cartridge or plate-and-frame structure comprising the filtration membrane.

The term “macromolecule” refers to a large molecule, generally having a molecular weight greater than about 1 kDa. Macromolecules typically comprise at least thousands of atoms and are commonly created by the polymerization of smaller subunits or monomers. Macromolecules of biological or synthetic origin are well known. Synthetic macromolecules include common plastics, synthetic fibers, carbon nanotubes and synthetic water soluble polymers, including both anionic and cationic polyelectrolytes. “Biological macromolecules” are macromolecules found in or associated with living organisms, including nucleic acids, proteins, carbohydrates and lipids, and composites of such molecules such as glycoproteins and lipoproteins. As used herein, the term “biological macromolecules” include complexes of more than one type of macromolecule, including viruses, and ribonucleoproteins (RNPs) such as ribosomes and small nuclear RNPs.

“Processing” refers to the act of filtering (e.g., by SPTFF) a feed containing a product of interest and subsequently recovering the product in a concentrated form. The concentrated product can be recovered from the filtration system (e.g., a SPTFF system) in either the retentate stream or permeate stream depending on the product's size and the pore size of the filtration membrane.

The term “protein” refers to a substance comprising at least one “polypeptide”, which herein means an amino acid polymer containing at least about eight constituent amino acid residues covalently joined by peptide bonds and having a molecular weight of at least about 1 kDa. The terms “polypeptide” and “peptide” are used interchangeably. “Multimeric proteins” contain two or more polypeptide chains held together by noncovalent bonds. The term “protein” includes protein composites such as glycoproteins and lipoproteins, and protein complexes such as nucleoproteins and ribonucleoproteins.

“Tangential flow filtration (TFF)”, a rapid and efficient method for separation, purification and concentration of biomolecules, is a process that uses membranes to separate components in a liquid solution or suspension on the basis of size, molecular weight or other differences. In traditional TFF processes, the fluid is pumped tangentially along the membrane surface and particles or molecules which are too large to pass through the membrane are rejected and returned to a process tank for additional passes across the membrane (e.g., recirculation) until the process fluid is sufficiently concentrated or purified. The cross-flow nature of TFF minimizes membrane fouling, thus permitting high volume processing per batch.

The term “Single-Pass TFF (SPTFF)” refers to a TFF process that allows direct flow-through concentration of a product (e.g., a biological macromolecule) in the absence of recirculation, which reduces overall system size and permits continuous operation at high conversion levels. The expression “conversion” refers to the fraction of the feed volume that permeates through the membrane in a single pass through the flow channels, expressed as a percentage of the feed stream volume. Compared with traditional recirculating TFF, SPTFF runs at constant operating conditions throughout the process, simplifies the required hardware, allows higher concentration factors and higher product recovery without significant dilution by reducing hold-up volume, and reduces the risk of product damage.

The terms “SPTFF apparatus” and “SPTFF system” are used interchangeably herein to refer to a TFF assemblage that is configured for operation in a single-pass TFF mode. A “single-pass TFF mode” refers to operating conditions for a TFF apparatus under which all or a portion of the retentate is not recirculated through the system.

An “in-series” SPTFF system refers to one that allows “serial processing” of the solution undergoing concentration. Such a system comprises a plurality of cassettes that are fluidly connected by distributing the feed directly from the feed channel to only the first processing unit in the assembly. In serial processing, each of the other, subsequent processing units in the assembly receives its feed from the retentate line of the preceding processing unit (e.g., the retentate from a first processing unit serves as the feed for a second, adjacent processing unit).

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about” refers to a numeric value that is within an acceptable error range for that particular value as determined by one of ordinary skill in the art, which will depend in part on how the value is measured or determined, i.e., the limitations of the measurement system. For example, “about” can mean within 1 or within more than 1 standard deviation per the practice in the art. Alternatively, it can mean a range of plus or minus 20%, more usually a range of plus or minus 10%. When particular values are provided in the application and claims, unless otherwise stated, the meaning of “about” should be assumed to be within an acceptable error range for that particular value.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Theory Underlying SPTFF

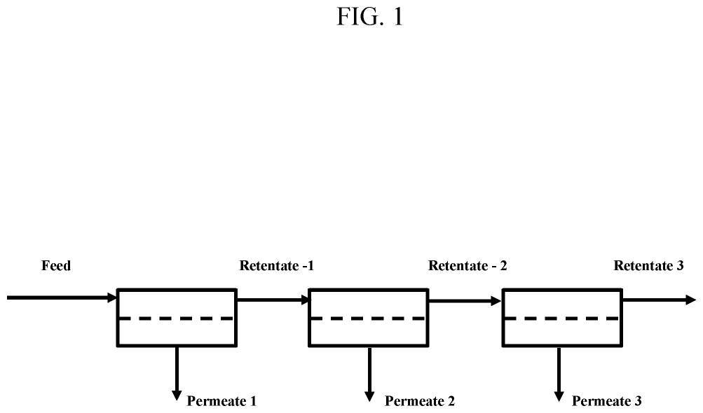

SPTFF is a membrane technology that has become commercially available in recent times to enable concentration of biomolecules, e.g., proteins, in a single pump-pass through the module feed channels. This single pass concentration is achieved by reducing the feed flow rate into the module and allowing enough contact time of the biomolecule solution within the module to allow high conversion of the feed solution into the permeate. The length of the module is increased, the modules are arranged in series, and the area of the membrane is consequently increased to allow higher conversions in a single pump pass (FIG. 1). Two different approaches to staging are possible: the unequal area staging (also called the Christmas Tree staging), and the equal area staging. The basic premise to increase the overall membrane area and the module length is the same for both these approaches to device design. The experiments described herein employed only equal area staging.

In ultrafiltration, the partially and completely retained solutes accumulate at the wall of the membrane and form a polarized boundary layer. This phenomenon is called concentration polarization and the concentration of the solute (e.g., protein) at the wall, C_{W} is related to that at the bulk, C_{b} by means of Equation 1.

$${C}_{W} = {C}_{b} ( {S}_{o} + ( 1 - {S}_{o} ) exp ( \frac{J}{k} ) ) \qquad Equation 1$$

where J is the filtrate flux, k is the boundary layer mass transfer coefficient and Sₒ is the observed sieving coefficient given by Sₒ=Cₚ/C_{b}, where Cₚ is the concentration of solute (e.g., protein) in the permeate. In a system where the membrane is completely polarized and the polarized boundary layer controls the separation, the wall concentration should have a constant value at a given axial position, z, and it will change (increase) throughout, depending on the filtrate flux and the protein concentration at each z. This means that as long as the feed solution to the membrane module is the same, the wall concentration at each z is constant (pressure-independent ultrafiltration), the filtrate flux and the retentate concentration are constant at each axial point, allowing for constant operating conditions throughout the process.

When the solute is partially retained as, for example, some applications for protein fractionation or permeation may require, the sieving coefficient will depend on the wall concentration. It is well known in ultrafiltration that increasing the wall concentration increases the sieving coefficient for pure protein solutions in buffer (Lebreton et al. 2008; Ruanjaikaen and Zydney 2011).

Strictly speaking, the sieving coefficient of a partially retained protein is not constant in SPTFF since the protein concentration changes throughout the length of the module. Furthermore, it is difficult to measure the sieving coefficient in SPTFF because of changing hydraulics at each point in the module. Nonetheless, it is possible to break down the SPTFF system into three-stages, exactly as it is assembled, and an “average” sieving coefficient can be estimated based on the permeate and retentate concentrations at each stage using the equations presented by Arunkumar and Etzel (2013) according to Equation 2:

$$\overline{{S}_{o}} = 1 - ln ( \frac{{C}_{R , i}}{{C}_{R , i - 1}} ) / ln {VCF}_{i} \qquad Equation 2$$

where C_{R,i} is the retentate coming out of stage i, C_{R,i-1} is the retentate exiting the (i−1)^{th} stage (thereby becoming the feed to the i^{th} stage), VCFᵢ is the volume concentration of the i^{th} stage. The overall mass balance of the system is given by Equation 3:

C_{F}= [symbol] C_{P}+(1− [symbol] )C_{R} Equation 3:

[symbol]

is the total conversion of the feed to the permeate (given by [symbol] =Q_{P}/Q_{F}), C_{P} is the overall permeate concentration and C_{R} is the overall retentate concentration.

The overall mass balance can be used to calculate an “overall average” sieving coefficient, [symbol] S_{O} [symbol] using Equation 2 and Equation 3 according to Equation-4 to achieve an overall average VCFₜ:

$$〈 {S}_{o} 〉 = 1 - ln ( \frac{{C}_{R}}{{C}_{F}} ) / ln ( VC {F}_{t} ) \qquad Equation 4$$

The mAbs (molecular mass=140-150 kDa) employed in the present studies were completely retained by the 10 kDa composite regenerated cellulose (CRC), 30 kDa composite regenerated cellulose (CRC) and 50 kDa polyethersulfone (PES) membranes. Thus, Sₒ was set to Sₒ=0 for the retained Abs. Lysozyme was partially retained by these membranes (0<Sₒ<1). While it is straightforward to understand lysozyme behavior using conventional TFF by measuring the sieving coefficient as a function of polarization conditions (filtrate flux and crossflow rate), the above equations are necessary to estimate the sieving behavior of lysozyme in each section of the SPTFF module.

### Comparison of Sieving Coefficients of Lysozyme Using TFF and SPTFF

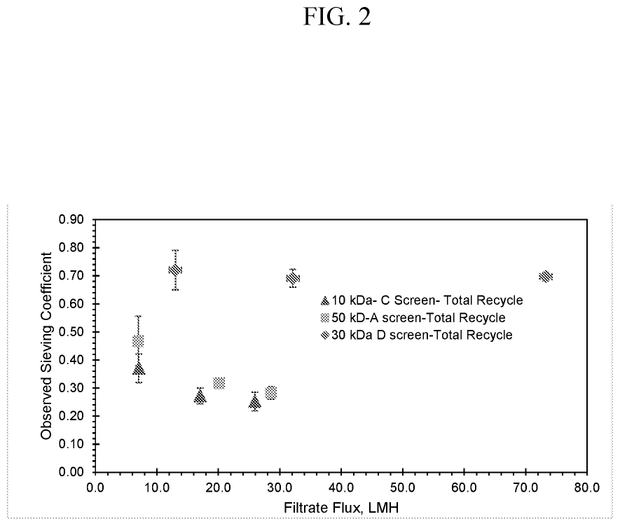

The sieving behavior of a partially retained protein, lysozyme, was investigated in TFF as described in Example 2, and in SPTFF as described in Example 3. The trend in the sieving coefficients of lysozyme as a function of flux using the TFF mode at a normalized feed flow rate of 100 L/h/m² was expected, but the sieving behavior of lysozyme as a function of MWCO was surprising. The flow rate of 100 L/h/m² was chosen because TFF is typically operated at a normalized feed flow rate of 200-800 L/h/m², and 100 L/h/m² was a normalized feed flow rate that represented polarization conditions more typical of SPTFF. The TFF data shown in FIG. 2 indicate that the 10 kDa CRC and the 50 kDa PES membranes exhibited very similar sieving behavior towards lysozyme, whereas the 30 kDa CRC membrane had the highest sieving coefficients. This agrees with the finding by Bakshayeshi et al. (2012) that the rating of membranes using dextran retention tests is not yet a standardized practice, and different vendors rate their membranes differently. Nevertheless, the 10 kDa CRC, 30 kDa CRC and 50 kDa PES membranes were all manufactured by MilliporeSigma, and the MWCO rating based on dextran sieving is expected to hold true for proteins as well, regardless of the membrane material. The choice of the material (CRC versus PES) is based on its compatibility with the solution to be ultrafiltered. A 50 kDa CRC membrane would have had a higher permeability than a 10 kDa or a 30 kDa CRC membrane, but a 50 kDa CRC membrane was not commercially available from any vendor that manufactures ultrafiltration membranes.

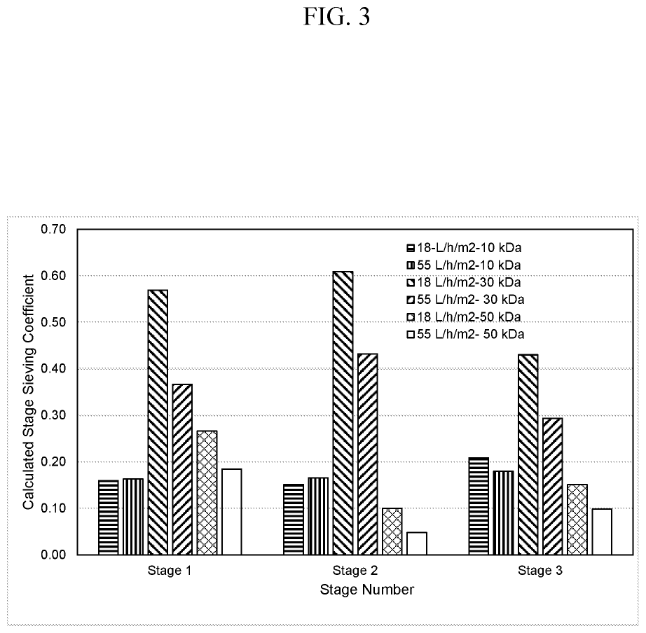

The trend for SPTFF (Example 3) somewhat qualitatively agrees with the classical stagnant film model: decreasing the feed flow rate increases the residence time of the protein in the retentate channel and hence the concentration at every section of the SPTFF module. This increases the accumulation of lysozyme at the membrane wall, C_{W}, and results in a higher sieving coefficient (Equation 1). This is very apparent for the 30 kDa CRC membrane where the sieving coefficient increased at stage 2 compared to stage 1, but not so for the 10 kDa CRC and the 50 kDa PES membranes (FIG. 3). In fact, the sieving coefficients of the 50 kDa PES membrane decreased as a function of the stage, as the protein concentration increased through the module whereas that of the 10 kDa CRC membrane did not change as a function of the stage. The fact that decreasing normalized feed flow rate increases the residence time and hence the wall concentration is reflected in the trend of stage-wise sieving coefficients being higher at 18 L/h/m² compared to 55 L/h/m² for both the 30 kDa and 50 kDa membranes. The trend of decreasing sieving coefficients using the 50 kDa membrane as a function of the stage at a given feed flow rate was attributed to membrane fouling. The 50 kDa PES membranes had to be cleaned thoroughly using 400 ppm of bleach in 0.1M NaOH to restore the permeability.

The classical stagnant film model cannot sufficiently explain the differences in the sieving coefficients for SPTFF and the TFF mode. According to the classical stagnant film model, the sieving coefficient observed during TFF (FIG. 2) should be the lowest, since the feed flow rate is the highest (100 L/h/m² compared to 55 L/h/m² and 18 L/h/m² for SPTFF) and the concentrations are lower and more uniform (10 mg/mL for TFF versus an increasing concentration in SPTFF). This means that the wall concentration should have been lower at every flux tested for TFF, and hence the sieving coefficients also lower. The reverse is observed: The sieving coefficient is highest using the TFF mode for all the membranes. The differences in the sieving coefficients could be attributed to differences in the mechanism of concentration polarization using SPTFF versus conventional TFF. Moreover, a more complex dependence on protein concentration is indicated by the data in FIG. 3 for the 30 kDa CRC membrane, where the sieving coefficient decreases with increasing concentration in stage 3, regardless of the normalized feed flow rate.

This behavior indicates that the ultrafiltration behavior of partially retained solutes may be complicated using SPTFF compared to TFF, and any process for separating macromolecular solutes using SPTFF may not be able to depend on extrapolation of the sieving behavior expected from TFF or stirred cells. Examples of such separations of partially retained solutes include microfiltration of clarified harvest (Bolton et al. 2017), separation of individual proteins from bioprocess streams (Lebreton et al. 2008; Arunkumar and Etzel 2013), separation of PEGylated proteins from PEG and non-PEGylated proteins (Ruanjaikaen and Zydney 2011; Kwon et al. 2008), ultrafiltration of other therapeutic modalities like viral vectors and plasmid DNA, and fractionation of dairy protein fractions in the food industry (Arunkumar and Etzel 2013; Arunkumar and Etzel 2014). These separation processes to obtain proteins in the permeate rely on concentration polarization to boost the separation.

The data also indicate that a modified concentration polarization model would be required to be developed for SPTFF, and recirculation-TFF or stirred cell behavior cannot be conveniently leveraged to be used in SPTFF.

The sieving behavior of lysozyme using TFF and SPTFF was an unexpected finding: it was expected that the 10 kDa CRC membrane would be much tighter towards lysozyme and the 50 kDa PES membrane would be most open to lysozyme, with the 30 kDa CRC membrane being intermediate between the two. In fact, the hydraulic permeability of the 50 kDa PES membrane was the highest (L_{P}=425±20 LMH/bar), compared to the 30 kDa CRC (L_{P}=142±18 LMH/bar) and 10 kDa CRC membranes (L_{P}=98±4 LMH/bar). The permeability and the rating of the membrane as “50 kDa” by themselves suggested that the 50 kDa membrane would be completely permeable to lysozyme. This could be attributed to the differences in structure between the BIOMAX® and ULTRACEL® membranes, and also in the method of rating these membranes (Bakhshayeshi et al. 2012). Recent work by Manzano and Zydney (2017) demonstrate similar differences in results for RNA transmission through 100 kDa CRC and PES membranes. This important observation warrants future investigation in the context of SPTFF, because the nominal MWCO as “50 kDa” is misleading since it was comparable to the 10 kDa with regard to sieving behavior but had a higher permeability, indicative of a more open membrane. This observation also calls the methodology of rate ultrafiltration into question: current methods for rating membranes are agnostic regarding the membrane surface chemistry. With the industry expanding the use of ultrafiltration and membrane technology, more rigorous characterization techniques will be required to accurately rate ultrafiltration membranes.

### Behavior of Completely Retained Proteins (MAbs) Using SPTFF

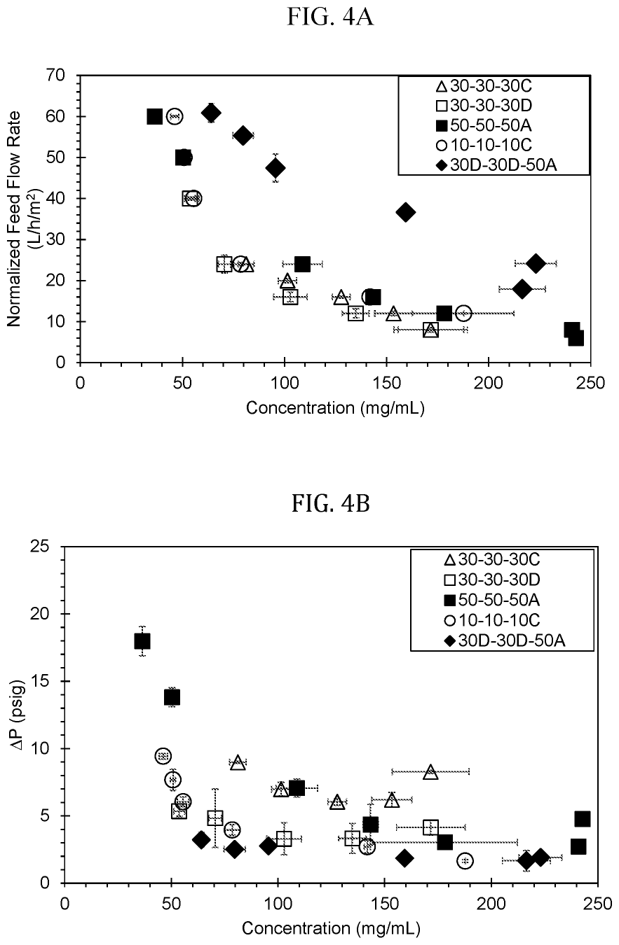

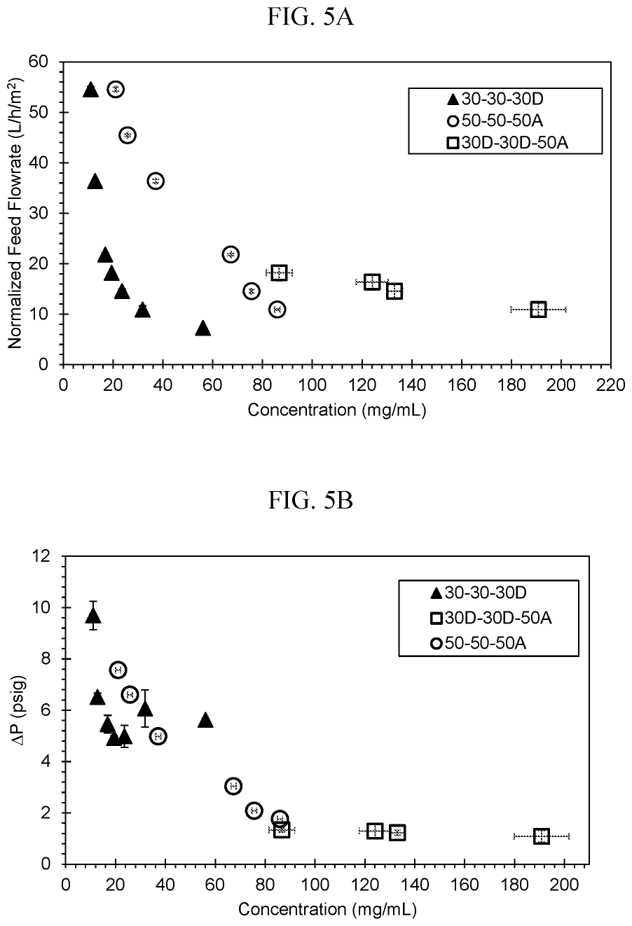

Solutions of two mAbs, mAb1 and mAb2, were concentrated by SPTFF. The data presented in FIG. 4 and FIG. 5 are significant when viewed in the context of results published by several groups on highly concentrated protein solutions (Binabaji et al. 2016; Baek et al. 2017; Thiess et al. 2017), in which the module screen type and the buffer composition significantly affected the ability to reach high concentrations for BSA, mAbs and Fc fusion proteins. The general conclusion from all these studies is that the axial pressure drop in TFF cassettes causes reverse filtration at high protein concentrations because of loss of retentate pressure at the module exit. Furthermore, the intermolecular interactions between highly concentrated mAbs was strongly influenced by the type of buffer used: the viscosity and osmotic pressure effects were significantly different and higher in a histidine matrix compared to a phosphate matrix (Baek et al. 2017). The data shown in FIG. 4 for mAb1 using SPTFF indicate that the volume concentration factor was not affected by the buffer matrix, the membrane screen type or the molecular weight cut-off between 10 kDa and 30 kDa for CRC membranes. Higher flow rates were possible using a 50 kDa membrane and the 30-30-50 kDa configuration, which was likely related to the higher permeability of the 50 kDa PES membrane and the non-uniform pressure drop from the 30-30-50 kDa configuration. The wall concentration will increase throughout the module, with the intermolecular interactions arising from buffer and protein interactions becoming significant only at the final sections where flow rates are already low and the retentate pressure is still finite (non-zero), giving a positive transmembrane pressure (TMP), in contrast to conventional TFF, where the osmotic pressure contributions result in reverse flow at the module exit.

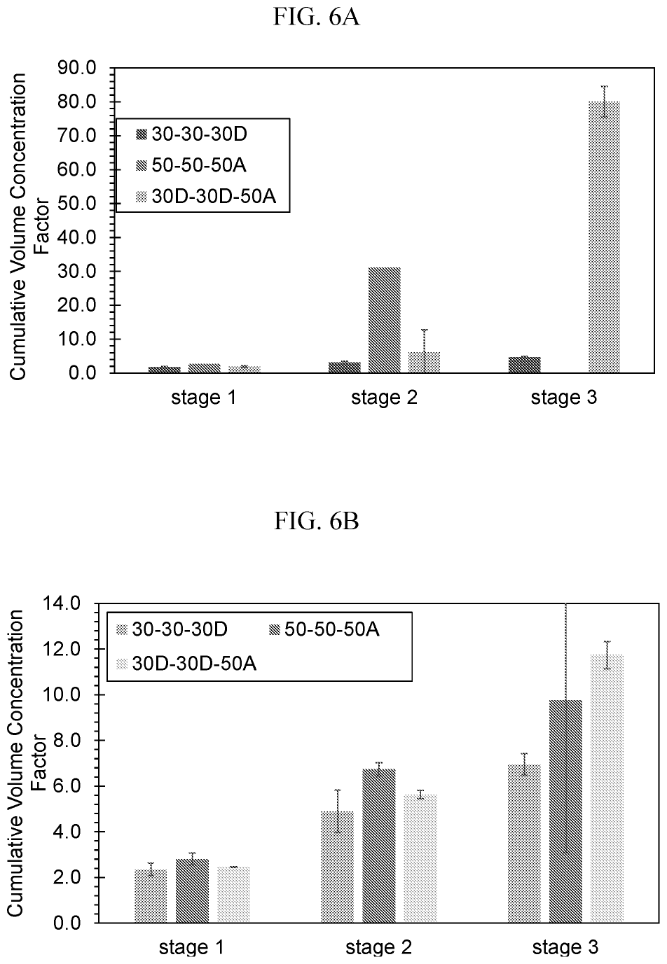

Taken together, the data indicate that the 10 kDa and 30 kDa membranes functioned similarly and it did not matter which of the two was used in single pass concentration for retentive mAbs. The screen type and buffer composition did not affect the maximum concentration achieved. However, there was a significant difference between the 30 kDa and 50 kDa membranes, with the 50 kDa or the 30-30-50 hybrid configurations providing higher normalized feed flow rates to achieve the same target concentrations. A complete 1 h concentration performed using all these membranes also indicated that the 30-30-50 kDa hybrid configuration was the most stable in terms of consistency. The 50 kDa system alone required lesser area than the 30 kDa system (0.22 m² compared to 0.33 m²) to achieve a given target concentration. However, the 50 kDa system (50-50-50 A) by itself also exhibited inconsistent performance; membrane fouling became an issue during concentration and the process had to be interrupted when concentrated beyond 1 h. The inconsistency for the 50 kDa membrane is reflected in FIG. 6 in the high standard deviation for the volume concentration factor. This high standard deviation for the 50 kDa membrane for protein concentration reflects the change in protein concentration during the 30 min measurement time period, and also a variability that resulted from the fouling of the more open membrane. The 30 kDa system and the 30-30-50 kDa systems were tested to operate up to 4 h without any change in hydraulics or conversions (protein concentrations) for both mAb1 and mAb2. Material limitations prevented operating for longer periods.

The 30-30-50 kDa system did not foul in between runs and was able to operate at a higher flow rate compared to the 30 kDa membrane system or the 50 kDa membrane system alone. The data shown in FIG. 6 illustrate what happens to the cumulative volume concentration factor in different stages of the SPTFF. The first two stages perform the majority of the conversion. As the concentrated protein enters the third stage, it is highly concentrated and approaches the wall concentration, C_{W}, in the last stage for the 10 kDa and 30 kDa membrane, limiting the maximum achievable concentration realistically. When the third stage was replaced with a 50 kDa membrane, the first two stages performed the majority of the conversion, but the 50 kDa membrane in the third stage was more permeable, had a higher wall concentration, and increased the concentration maximum to a greater degree than either the 10 kDa or the 30 kDa membrane. Moreover, the higher permeability of the 50 kDa membrane allowed operation at 2-4 times higher normalized feed flow rates than the 30 kDa membrane alone, reducing the processing time significantly. The ability of the hybrid system to achieve very high volume concentration factors (approximately 80× for mAb2 at a 57% higher normalized feed flow rate compared to the 30 kDa membrane) and operate at significantly higher feed flow rates is a very important outcome of this study.

Based on the experimental studies described in the Examples and discussed above, the present application provides a method for concentrating a solution of a macromolecule that is retained on at least two semi-permeable membranes that have different molecular weight cutoffs (MWCOs), the method comprising passing the solution through a hybrid configuration of said semi-permeable membranes staged in series in a SPTFF apparatus, wherein the last membrane in the series has a larger MWCO than the preceding membrane or membranes. This method is exemplified herein (Example 4) to concentrate mAb solutions using three filtration membranes having two different MWCOs, 30 kDa and 50 kDa, and staged in series in a hybrid 30-30-50 kDa configuration. However, a person skilled in the art would readily appreciate that this method is not limited to the use of three membranes; for example, two membranes in a 30-50 kDa configuration, or four membranes in a 30-30-30-50 kDa configuration, or even five or more membranes, could be used for the concentration of these mAbs.

In certain embodiments of this method, the macromolecule is a biological macromolecule. In certain further embodiments, the biological macromolecule is chosen from a protein, nucleic acid, DNA, oligonucleotide, RNA, virus particle, ribonucleoprotein, carbohydrate, glycoprotein, lipid, triglyceride, phospholipid, lipoprotein, and a fragment or portion of any of said biological macromolecules. DNA includes, for example, chromosomal DNA, genomic DNA, cDNA, viral DNA, expression vector DNA, plasmid DNA, viral vector DNA, vaccine DNA, deoxyribonucleotides, RNA, and ribonucleotides. In further embodiments, the protein is chosen from a polypeptide, a multimeric protein, a therapeutic protein, an antibody, an antigen-binding portion of an Ab, an antibody-drug conjugate, an immunoconjugate, an Fc portion of an Ab, an Fc fusion protein, a glycoprotein, a lipoprotein, a deoxyribonucleoprotein such as a nucleosome or a DNA virus, a ribonucleoprotein (RNP) such as a ribosome, a small nuclear RNP (snRNP) or a RNA virus, a PEGylated protein, and a fragment or portion of any one of these proteins.

In certain other embodiments, the biological macromolecule has a molecular weight of about 3 to about 10, about 10 to about 20, about 15 to about 30, about 20 to about 40, about 40 to about 60, about 60 to about 90, about 30 to about 90, about 90 to about 120, about 90 to about 150, about 120 to about 180, about 150 to about 300, about 300 to about 900, about 900 to about 1800, or greater than about 3,000 kDa. Examples of biological macromolecules, specifically proteins, and their corresponding sizes, are shown in Table 1. Such proteins can be concentrated by the present methods using a hybrid configuration of filtration membranes having appropriate MWCOs.

TABLE 1
Molecular masses of representative proteins^{a}

| | Molecular Mass of | Molecular Mass of |
|---|---|---|
| Protein | Polypeptide (kDa) | Native Protein (kDa) |
| Aprotinin | 6.5 | |
| Chicken lysozyme | 14.3 | |
| Carbonic anhydrase | 29.0 | 180.0 |
| Ovalbumin | 45.0 | |
| IgG heavy chain | 55.0 | 150.0 |
| Human transferrin | 80.0 | 80.0 |
| Phosphorylase-b | 94.0 | |
| IgG antibody | 140-150 | |
| α₂-Macroglobulin | 170.0 | 820.0 |
| Myosin | 205.0 | 470.0 |

^{a}Data obtained from Proteins used as molecular-weight standards - Proteins and Proteomics, online website (http://www.proteinsandproteomics.org/content/free/tables_1/table10.pdf)

In certain preferred embodiments of the present methods, three semi-permeable membranes, each contained within a cassette, are used in the SPTFF apparatus. In other embodiments, two or four semi-permeable membranes are used in the SPTFF apparatus.

It is important to select the appropriate MWCO for the ultrafiltration membranes. MWCOs are nominal ratings based on the ability of the membrane to retain greater than 90% of a solute of a known molecular weight (in kDa). The retention characteristics of different MWCO membranes are known for different solutes such as nucleic acids, proteins, and virus particles (see, e.g., Pall Corporation Selection Guide: Separation Products for Centrifugal and Tangential Flow Filtration, available online). For proteins, it is recommended that a MWCO be selected that is about three to about six times smaller than the molecular weight of the solute being retained. However, because different manufacturers use different molecules to define the MWCO of their membranes, it is important to perform pilot experiments to verify membrane performance in a particular application. A section of MWCOs is shown in Table 2 for different proteins.

TABLE 2
MWCO selection for protein SPTFF applications

| MWCO (kDa) | Protein Molecular Mass (kDa) |
|---|---|
| 1 | 3-10 |
| 3 | 10-20 |
| 5 | 15-30 |
| 10 | 30-90 |
| 30 | 90-180 |
| 50 | 150-300 |
| 100 | 300-900 |
| 300 | 900-1,800 |
| 1,000 | >3,000 |

Other factors may be considered in choosing the MWCO. For example, if reducing flow rate is a consideration, a membrane with a MWCO at the lower end of this range (3×) is chosen; conversely, if retention of the solute is a major concern, a tighter membrane (6×) is chosen. Retention of a molecule by an ultrafiltration membrane is determined by a variety of factors, among which its molecular weight serves only as a general indicator. Therefore, choosing the appropriate MWCO for a specific application requires the consideration of other variables, including molecular shape, electrical charge, sample concentration, sample composition, and operating conditions.

Based on the above considerations and the data disclosed herein, a person skilled in the art will be able to select appropriate MWCOs for assembling an appropriate hybrid configuration from a wide variety of membrane options for use in the present methods, as the following non-limiting examples illustrate. In certain embodiments, the biological macromolecule, such as a protein, has a molecular weight of about 90 to about 180 kDa and the membranes are staged in a 20-30, a 20-40, a 25-40, a 25-50, a 30-50, a 20-20-30, a 25-25-40, a 30-30-50, a 20-30-40-50, a 20-20-20-40, a 25-25-25-40, or a 30-30-30-50 kDa hybrid configuration. In certain preferred embodiments of this molecular weight class, the biological macromolecule is an antibody and the membranes are staged in a 30-30-50 kDa hybrid configuration.

In certain other embodiments, the biological macromolecule, such as a protein, has a molecular weight of about 30 to about 90 kDa and the membranes are staged in a 5-10, a 5-5-10, a 8-12, a 8-8-12, a 10-15, a 10-10-15, a 12-15, a 12-12-15, a 15- 20, a 15-15-20, a 20-30, a 20-20-30, a 15-15-15-20, or a 20-20-20-30 kDa hybrid configuration. In further embodiments, the biological macromolecule, such as a protein, has a molecular weight of about 10 to about 30 kDa and the membranes are staged in a 3-5, a 3-3-5, a 5-8, a 5-5-8, a 5-5-10, a 8-8-10, a 3-5-8-10, a 5-5-5-10, or a 8-8-8-10 kDa hybrid configuration. For example, for concentrating a solution of lysozyme (molecular weight 14.3 kDa) by the present SPTFF methods, a person skilled in the art could reasonably select a hybrid 3-5, 3-3-5, 3-5-8, 5-5-8 or a 3-3-5-8, or a 5-5-5-8 kDa configuration. In certain preferred embodiments for concentrating a solution of lysozyme, the membranes are staged in a 5-5-8 kDa hybrid configuration.

In certain embodiments, electrically charged membranes are also employed in the present methods. For example, using a negatively charged membrane with a 1,000 kDa MWCO (designated “1,000(−)”) to concentrate a macromolecule such as a monoclonal antibody having a negative charge at pH 6 would cause the antibody to be electrostatically repelled by the membrane. Accordingly, in certain embodiments, a high antibody concentration is achieved using a 30-1000(−) kDa configuration at a significantly higher feed flow rate than by using the 30-30-50 configuration. The use of electrically charged membranes can be similarly incorporated into the claimed methods for macromolecules of different size ranges, as would be evident to a person of ordinary skill in the art.

As described in Example 4, use of the 30-30-50 kDa configuration significantly pushed the maximum concentration of the mAb2 antibody beyond 150 mg/mL to as high as about 200 mg/mL (FIG. 5). Accordingly, in certain embodiments of the disclosed methods for concentrating an antibody solution, the method achieves a concentration of about 150 mg/mL. In certain other embodiments, the method achieves a concentration of about 75 to about 100, or about 100 to about 150 mg/mL. In further other embodiments, the method achieves a concentration of about 150 to about 200 mg/mL. Other hybrid configurations, e.g., a 25-25-25-40 or a 30-30-30-50 kDa configuration, may achieve even higher concentrations. Thus, in certain other embodiments, the method achieves a concentration of greater than about 200 mg/mL, for example, a concentration of about 200 to about 250, or about 250 to about 300 mg/mL.

Example 4 also demonstrates that a 30-30-50 kDa configuration of membranes achieved a concentration 12× higher than the concentration of the starting solution for mAb1 and about 80× higher for mAb2 (FIG. 6). These increased concentrations were achieved notwithstanding that flow rates were 183% higher for the 30-30-50 kDa configuration for mAb1 compared to the 30-30-30 D configuration, and 57% higher for the 30-30-50 kDa configuration for mAb2, compared to the 30-30-30 D membrane configuration. Thus, in certain embodiments of the disclosed methods for concentrating a macromolecule, such as an antibody, the method achieves a concentration of about 12-fold or at least about 12-fold higher than the concentration of the starting solution. In certain other embodiments, the method achieves a concentration of about 80-fold or at least about 80-fold higher. In other embodiments, the method achieves a concentration of about 100-fold higher. In yet other embodiments, the method achieves a concentration of about 150-fold higher. In further embodiments, the method achieves a concentration of about, or at least about, 5, 10, 15, 20, 30, 50, 60, 70, 75, 90, 100, 150, or greater than 150-fold higher than the concentration of the starting solution.

In certain other embodiments for concentrating a macromolecule, such as an antibody, the hybrid configuration of two different membranes (e.g., 5-5-8 kDa, or 30-30-50 kDa) allows operation at a flow rate about, or at least about, 2-fold higher than the maximum flow rate achieved using the same membranes in a non-hybrid configuration (e.g., 5-5-5, 8-8-8, 30-30-30 or 50-50-50 kDa). In certain embodiments, the hybrid configuration allows operation at a flow rate about, or at least about, 4-fold higher than the maximum flow rate achieved using the same membranes in a non-hybrid configuration. In certain embodiments for concentrating an antibody solution, a hybrid 30-30-50 kDa configuration allows operation at a flow rate about, or at least about, 2-fold higher than the maximum flow rate achieved using membranes in a 30-30-30 kDa or 50-50-50 kDa configuration. In further embodiments for concentrating an antibody solution, a hybrid 30-30-50 kDa configuration allows operation at a flow rate about, or at least about, 4-fold higher than the maximum flow rate achieved using membranes in a 30-30-30 kDa or 50-50-50 kDa configuration.

This application describes the single-pass ultrafiltration behavior of partially and completely retained proteins, exemplified by lysozyme and two different mAbs, respectively, using 10 kDa CRC, 30 kDa CRC and 50 kDa PES membranes. Whereas these types of proteins were found to behave differently in terms of sieving, the data from both these types of proteins have been combined to reach several conclusions. Firstly, the sieving behavior of lysozyme using 10 kDa CRC and 50 kDa PES membranes was similar in the total recycle TFF mode. The 30 kDa CRC membrane gave an approximately 2.8× higher sieving coefficient (Sₒ=0.70 at J_{V}=30 LMH) compared to the 10 kDa CRC membrane (Sₒ=0.25 at J_{V}=26 LMH) or the 50 kDa PES membrane (Sₒ=0.28 at J_{V}=28 LMH). If a separation were to be performed to separate a solute like lysozyme from a larger protein, the data using total recycle experiments would suggest using the 30 kDa CRC membrane to be optimal. However, data obtained from SPTFF experiments suggest that the sieving coefficients can be about 1.6-2.5× lower depending on the feed flow rate ((Sₒ)=0.55±0.04 at a feed flow rate of 18 L/h/m² and (S₀)=0.37±0.03 at a feed flow rate of 55 L/h/m² for the 30 kDa membrane) (Table 5), indicating that the process to perform the separation would require either more open membranes or an optimization study to find the ideal feed flow rate and the ideal configuration. The sieving coefficients measured and calculated using SPTFF were also more sensitive compared to that measured using TFF. The 10 kDa CRC membranes and the 50 kDa PES membranes did not differ significantly irrespective of the mode of operation (TFF versus SPTFF). Thus, the 50 kDa PES membrane would be a better option than the 10 kDa CRC membrane considering the flux increase that the 50 kDa PES membrane offered at the same or better retention.

With the biopharma industry moving towards continuous bioprocessing, and with other emerging therapeutic modalities that necessarily require a single pump pass through microfiltration and ultrafiltration membranes for fractionation, this is a significant finding that will impact such separations. An example of such a separation process valid in the context of the data disclosed herein would be the use of single pass TFF to separate PEGylated (or conjugated) proteins from unreacted PEG using ultrafiltration. The viscosity of the PEGylated proteins could be high, limiting the use of TFF mode, but allowing the use of an SPTFF configuration. With the implementation of a continuous diafiltration strategy (Nambiar et al. 2018) and using the correct membrane configuration, this separation may be performed using SPTFF. However, the choice of the membrane and expected sieving behavior from TFF experiments will have to be reevaluated in terms of the data presented herein.

For completely retained solutes, the conversions were much more predictable. As expected, the 50 kDa PES membrane was more permeable and allowed higher conversions. The 50 kDa system alone led to overconcentration in the first and second stages, leading the third stage to be redundant. However, a tight control on the feed flow rate was difficult to obtain, and the 50 kDa membranes by themselves fouled severely. This is reflected in the larger error bars for protein concentration and conversion (FIG. 6). The 30-30-50 kDa system allowed operation between 2-4× higher feed flow rates and higher conversions. In addition to using the hybrid approach for protein concentration alone, such an approach may be effective for selective fractionation of proteins by tailoring the sieving coefficients to the desired value.

The data disclosed herein are the first from any study of the behavior of a partially retained solute like lysozyme and a completely retained solute like a mAb using commercially available, equal area-staged SPTFF modules. The sieving coefficients measured using TFF and SPTFF indicate a more complicated concentration polarization behavior for SPTFF compared to TFF. The retention of lysozyme was higher using SPTFF compared to TFF for all the types of membranes. This observation is counter-intuitive in the context of the classical stagnant film model. In the case of retained solutes like mAbs, the 10 kDa and 30 kDa membranes gave the same conversions at the same feed flow rates and the screen type made no significant change. Moreover, the effects of the buffer matrix were not significant for the SPTFF mode. The low flow rates required to achieve high conversions were improved by hybridizing the module with a 50 kDa membrane as the third stage after two 30 kDa membranes. The overall results of this study indicate that SPTFF is an attractive process for concentration of macromolecules, such as proteins, in a single pump pass.

The present invention is further illustrated by the following examples which should not be construed as further limiting.

### EXAMPLES

The purpose of the studies described herein was to experimentally understand the single-pass ultrafiltration behavior of partially permeable proteins (hen egg white lysozyme, MW=14.3 kDa) and completely retentive proteins like mAbs (MW=140-150 kDa; the two specific mAbs used are proprietary Bristol-Myers Squibb mAbs), and to develop methods for enhancing the concentration of macromolecules using SPTFF. To this end, experiments were performed using membrane modules with molecular weight cutoffs of 10 kDa, 30 kDa and 50 kDa, and with different turbulent promoters (feed screens). Modules with different turbulent promoters based on different differential pressure are commonly used for protein concentration to achieve concentration targets (Binabaji et al. 2016; Baek et al. 2017).

### Example 1

### Materials and Methods

### Proteins Studied

The single-pass ultrafiltration behavior of two mAbs and lysozyme was investigated in the present study. The two mAbs (mAb1 and mAb2) were IgG4 mAbs having molecular masses of 140-150 kDa and physical characteristics as summarized in Table 3. Hen egg white lysozyme was obtained from MilliporeSigma (L-6876) and dissolved in 20 mM sodium phosphate, 150 mM sodium chloride, pH 7.2, to achieve a protein concentration of 10 mg/mL. This particular buffer composition was chosen because it had a conductivity of 16 mS/cm, which was enough to overcome electrostatic exclusion effects for lysozyme that have been known to impact protein sieving through a 30 kDa ultrafiltration membrane (Burns and Zydney 2001).

Ultrafiltration Membranes

The 10 kDa, 30 kDa and 50 kDa membranes used were obtained from MilliporeSigma, and had different screens as shown in Table 4. The 10 kDa and 30 kDa membranes were made of ULTRACEL® composite regenerated cellulose, while the 50 kDa membrane was made of BIOMAX® modified polyether sulfone (PES). The 50 kDa PES membrane was used because this was the only commercial option for a cutoff beyond 30 kDa that would completely retain a mAb. A 50 kDa CRC membrane was not available commercially for use.

Pressure sensors were obtained from Pendotech Corporation, Nassau, N.J.

TABLE 3
Proteins used in this work and their physical properties

| | Isoelectric | Phosphate | Histidine | Molecular | Target |
|---|---|---|---|---|---|
| | Point | Buffer | Buffer | Mass | Concentration |
| Proteins | (pI) | Composition | Composition | (kDa) | (g/L) |
| mAb1 | 9.2-9.6 | 20 mM Sodium | 20 mM Histidine, | 140-150 | 200 |
| | | Phosphate | 200-260 mM | | |
| | | pH 7.0-7.4 | Sucrose | | |
| | | | pH 5.6-6.2 | | |
| mAb2 | 7.2-7.8 | 20 mM Sodium | 20 mM Histidine, | 140-150 | 75 |
| | | Phosphate | 200-260 mM | | |
| | | pH 7.0-7.4 | Sucrose | | |
| | | | pH 5.6-6.2 | | |
| Lysozyme | 11.4 | 20 mM Sodium | N/A | 14.3 | N/A |
| | | Phosphate, | | | |
| | | 150 mM Sodium | | | |
| | | Chloride, | | | |
| | | pH 7.4 | | | |

TABLE 4
Membrane modules used and their characteristics

| | | | | ΔP at hydraulic | |
|---|---|---|---|---|---|
| Membrane | Modular | Total | Screen | flow rate of | |
| Train | Arrangement | Area (m²) | Channel | 4 L/min/m² | Vendor |
| 10 kDa | 3 × 0.11 m²-10 | 0.33 | Type C, | 14 | MilliporeSigma |
| Ultracel | kDa membranes | | 515 μm | | |
| (CRC) | | | | | |
| 30 kDa-C, | 3 × 0.11 m²-30 | 0.33 | Type C, | 10 | MilliporeSigma |
| Ultracel | kDa membranes | | 515 μm | | |
| (CRC) | | | | | |
| 30 kDa-D, | 3 × 0.11 m²-30 | 0.33 | Type D, | 2 | MilliporeSigma |
| Ultracel | kDa membranes | | 610 μm | | |
| (CRC) | | | | | |
| 50 kDa-A | 3 × 0.11 m²-50 | 0.33 | Type A, | 17 | MilliporeSigma |
| Biomax | kDa membranes | | 420 μm | | |
| (PES) | | | | | |
| 30D-30D-50A | 2 × 0.11 m²-30 | 0.33 | Hybrid of the | 10 | MilliporeSigma |
| | kDa D screen | | D screen and | | |
| | membranes | | A screen | | |
| | followed by | | | | |
| | 1 × 0.11 m² 50 | | | | |
| | kDa A screen | | | | |
| | membrane | | | | |

### Measurement of Protein Concentrations

Protein concentration of pooled samples was measured using a DropSense 96 well plate system (Trinean, Genbrugge, Belgium). The sample (4 μL) was loaded onto a 96-well plate and absorbance at 280 nm was measured. Absorbance was converted to protein concentrations using the empirically determined extinction coefficient, assuming that the Beer-Lambert law was valid. The measurements from the pool samples was used to confirm measurements from the FlowVPE system (C Technologies, Bridgewater, N.J.).

### Example 2

### Tangential Flow Filtration Experiments Under Conditions of Total Recycle to Measure Lysozyme Sieving Coefficients

Conventional TFF in recirculation mode was performed for lysozyme to characterize the TFF sieving behavior for comparison to the sieving behavior in SPTFF mode. Sieving coefficients were measuring as previously described (Lebreton et al. 2008; Arunkumar and Etzel 2014). One membrane module with an area of 0.11 m² for the 10 kDa CRC, 30 kDa CRC and the 50 kDa PES membranes was used for these measurements.

A lysozyme solution (10 mg/mL) was prepared by dissolution in 20 mM sodium phosphate, 150 mM sodium chloride, pH 7.2, and was recirculated through the 10 kDa CRC (C screen, Catalog Number P3C10001), 30 kDa CRC (D screen, Catalog Number P3C030D01) or 50 kDa PES (A screen, Catalog Number P3B050A01) ultrafiltration membrane under conditions of total recycle, at a membrane area-normalized feed flow rate (or “feed flux”, referred to as normalized feed flow rate throughout this application) of 100 L/h/m². The control valve on the retentate was used to adjust the inlet pressure on the feed to 2.0 bar (30 psig). A pump on the permeate side was used to control the permeate flux to the desired value and samples were collected from the permeate tubing and retentate tubing at different filtrate fluxes to measure the protein sieving coefficient.

FIG. 2 shows the variation of lysozyme sieving coefficient as a function of filtrate flux at a normalized feed flow rate (crossflow rate) of 100 L/h/m² using different membranes, operated in the total recycle TFF mode. The data show that the sieving coefficients of lysozyme using the 10 kDa CRC and 50 kDa PES ultrafiltration membranes are identical (p>0.05), with the data for the 10 kDa CRC and 50 kDa PES ultrafiltration membranes, essentially lying on top of each other. The sieving coefficient of lysozyme (MW=14.3 kDa) using the 30 kDa CRC ultrafiltration membrane module was 150% higher than the 10 kDa CRC membrane and 120% higher than the 50 kDa PES membrane (p<0.05) at a comparable filtrate flux of 17 LMH. The sieving coefficients did not change with flux for the 30 kDa membrane, while the sieving coefficients decreased with flux from 7 LMH to 30 LMH for the 10 kDa and 50 kDa membranes and remained constant thereafter.

### Example 3

### Single Pass Tangential Flow Filtration of Lysozyme

A Pellicon 3™ Single-Pass TFF system was used with a filtration area of 0.11 m² per stage. A diverter plate (Catalog: XXSPTFF01) was placed in a Pellicon-2™ Mini holder (Catalog Number XX42PMINI), with a gasket in between to seal the feed and permeate channels. Then 0.11 m² Pellicon 3™ TFF cassettes were inserted after the first diverter plate, giving a three-in-series system with a total area of 0.33 m², with each cassette separated by a diverter plate. The assembly was torqued to 23 Nm using a torque wrench.

Single-pass concentration of lysozyme was performed using two normalized feed flow rates of 55 L/h/m² and 18 L/h/m² using the 10 kDa CRC, 30 kDa CRC and 50 kDa PES ultrafiltration membranes The retentate pressure was adjusted to provide at least a 5× concentration in a single pass. Permeate and retentate were collected from every stage and measured for protein concentration. Based on the protein concentrations and flow rates, average sieving coefficients were calculated using Equation 2 for each stage and for the overall module for a given membrane and a given feed flow rate, using Equation 4.

The sieving coefficients of lysozyme calculated using SPTFF were lower than those measured during TFF for all the membranes (p<0.05). Furthermore, the calculated sieving coefficients of lysozyme for the 10 kDa CRC membrane were constant through all the stages, and also did not change as a function of the normalized feed flow rate with <Sₒ>=0.17±12% CV (p>0.05) (FIG. 3 and Table 5).

TABLE 5
Summary of protein concentrations, sieving coefficients and distributions
in the permeate and retentate for the concentration of lysozyme using
different SPTFF modules. Data is presented as average ± SD.

| | | | Overall | | | |
|---|---|---|---|---|---|---|
| | Feed Flow | | Sieving | % | % | Retentate |
| Membrane | Rate | Overall | Coefficient | Distribution | Distribution | Concentration |
| Configuration | (L/h/m²) | Conversion | [symbol] Sₒ [symbol] | in Permeate | in Retentate | (g/L) |
| 10 kDa, | 55 | 0.81 | 0.17 ± 0.02 | 23.5 | 75.0 | 41.8 |
| (3 × 10 kDa, C) | 18 | 0.94 | 0.16 ± 0.01 | 37.2 | 61.9 | 117 |
| 30 kDa, | 55 | 0.87 | 0.37 ± 0.03 | 49.7 | 50.2 | 43.8 |
| (3 × 30 kDa, D) | 18 | 0.86 | 0.55 ± 0.04 | 61.6 | 37.4 | 29.4 |
| 50 kDa, | 51 | 0.86 | 0.14 ± 0.03 | 19.7 | 80.3 | 48.5 |
| (3 × 50 kDa, A) | 19 | 0.91 | 0.21 ± 0.01 | 37.1 | 66.6 | 62.9 |

In the case of the 50 kDa PES membrane, the stage-wise sieving coefficient of lysozyme increased with decreasing normalized feed flow rate (p<0.05) for all the stages. The same trend was observed for the 30 kDa CRC membrane (p<0.05). The stage-wise sieving coefficients of lysozyme reported using the 30 kDa CRC membrane were the highest compared to the 10 kDa CRC and the 50 kDa PES membranes.

While the stage-wise sieving coefficient of lysozyme using the 10 kDa CRC membrane did not change with the stages, or the normalized feed flow rate, the sieving coefficient of the 50 kDa membrane decreased from stage 1 to stage 3, with the sieving data between stage 2 and stage 3 being indifferent at a given normalized feed flow rate (p>0.05) (FIG. 3).

The stage-wise sieving coefficient of lysozyme using the 30 kDa CRC membrane went through a maximum with the values at stage-2 being the highest for a given feed flow rate. Nonetheless, the differences between stage 1 and stage 2 were small, with the sieving coefficients at stage-3 being 24% lower than stage-1 at 18 L/h/m² and 20% lower than stage-1 at 55 L/h/m².

The distribution of lysozyme in the permeate followed the exact trend of increasing overall sieving coefficients (S₀), with decreasing normalized feed flow rates (Table 5). The (S₀) for the 50 kDa PES membrane did not differ from the 10 kDa CRC at 55 L/h/m², but was 31% higher than the 10 kDa CRC membrane at 18 L/h/m² (p<0.05). The data set for all the membranes reported in FIG. 3 and Table 5 were highly reproducible with a coefficient of variation (% CV) of less than 10%.

### Example 4

### Ultrafiltration Behavior of Completely Retained Monoclonal Antibodies Using Different Modular Configurations

SPTFF experiments were performed as described by Arunkumar et al. (2017). The protein solution was pumped through the membrane module at different flow rates and a retentate pressure of 10.0-15.0 psig to begin the process, with the value being increased using a control valve as target concentrations increased. A retentate pressure of 10-15 psig was chosen to ensure that all the ultrafiltration experiments were performed in the pressure independent regime of the flux versus TMP plot, which was generated separately at different normalized feed flow rates. For subsequent reporting of the data, the feed pressure and retentate pressures are not separately reported because the system control used two parameters—the area normalized feed flow rate and the retentate pressure. It is noted that it is the difference in pressures, rather than their absolute values, which is important. The absolute values of the pressure varies depending on factors such as the tubing and screen type used, but the difference in pressure is inherent. The manipulation of the flow rate and retentate pressure set a system feed pressure to the inlet of the SPTFF system. The absolute values of the feed pressure or retentate pressure did not show a trend; however, the feed flow rate coupled with the differential pressure through the channel was sufficient to provide a trend with the volume concentration factor and describe the system hydraulics completely.

The retentate was connected to a highly sensitive inline protein concentration measurement system based on absorbance at 280 nm (FlowVPE) that gave the continuous output of the protein concentration on the retentate. Each data point corresponding a particular normalized feed flow rate was collected only after equilibrating the system at the given normalized feed flow rate for at least 30 min. The attainment of equilibrium and constant output was determined by the Pendotech pressure trace and the protein concentration trace on the FlowVPE as a function of time. The data points reported in this study did not show deviations from constant outputs in the 30 min during which the measurement was made and reported.

Any discrepancy in the measured outlet concentration was immediately investigated. The flow rate, feed pressure, retentate pressure and the corresponding concentration were noted before proceeding to a different normalized feed flow rate. Samples were collected from the permeate of each stage separately to analyze for any losses due to protein sieving into the permeate. The procedure was repeated for different modular configurations and different protein solutions in their respective buffer compositions.

The mAbs mAb1 and mAb2 were completely retained using all the membranes. Since SPTFF is primarily used to concentrate these protein solutions for producing high concentration formulations, the effect of membrane MWCO and the type of screen channel were examined. As shown in FIG. 4, the normalized feed flow rate versus protein concentration plots for the 10 kDa CRC membrane and 30 kDa CRC membrane for mAb1 were indistinguishable. The corresponding differential pressures for the 10 kDa CRC and 30 kDa CRC membranes were similar but not the same. Nevertheless, the MWCO between the 10 kDa and 30 kDa or the screen type did not affect the performance for mAb1. The 50 kDa PES membrane had a higher differential pressure, presumably because of the tight screen in the 50 kDa PES module.

Furthermore, it was also observed that using a 50 kDa PES membrane in the last stage in a 30-30-50 kDa configuration helped in pushing the maximum concentration further than just using 30 kDa membranes or 50 kDa membranes, and allowed operation at a flow rate that was three-fold higher compared to the 30 kDa membranes or the 50 kDa membrane alone, even though the differential pressures were the same as the 30 kDa membranes (FIG. 4).

Similar observations were made for mAb2, where the 30-30-50 kDa configuration significantly pushed the maximum concentration beyond 150 mg/mL to as high as about 200 mg/mL, even though the target required to be achieved during processing was only 75 mg/mL (FIG. 5). The 50 kDa PES membrane met the target of 75 mg/mL, but the 30-30-50 kDa hybrid system was able to operate at a higher feed flow rate to achieve the same concentration objectives as the 30 kDa or the 50 kDa membranes. The data in both FIGS. 4 and 5 were averaged for mAb1 and mAb2 in both phosphate and histidine buffers, indicating that the buffer matrix did not affect the capability of the equal area staging to achieve final concentration targets (p>0.05). Both these figures also indicated that the differential pressures were similar for both the phosphate and histidine buffers with a % CV on the differential pressure being <5%.

Concentration experiments for 1 h were performed for mAb1 and mAb2 using the 30 kDa, 50 kDa and 30-30-50 kDa hybrid system at the lowest normalized feed flow rate realistically possible (which means the retentate flow rate was measurable accurately). The permeate flow rates from each stage were measured to calculate the contribution of each stage. The stage-wise cumulative volume concentration factor data is presented in FIG. 6. The 30-30-50 kDa configuration was capable of achieving significantly higher concentration factors compared to the standard 30-30-30 kDa configuration: 12× for mAb1 and about 80× for mAb2, even though the flow rates were 183% higher for the 30-30-50 kDa configuration for mAb1 compared to the 30-30-30 D configuration, and 57% higher for the 30-30-50 kDa configuration for mAb2, compared to the 30-30-30 D membrane configuration.

In studies on any pressure-driven filtration operation like ultrafiltration, it is common to report the hydraulics as a function of protein concentration using the differential pressure between the feed and retentate (ΔP) and the feed flow rate to get the differential pressure. Whereas the ultrafiltration behavior of a given module and/or configuration can be described completely using these two metrics, it is operationally important to understand the absolute values of the retentate pressure or the feed pressure along with the ΔP. The absolute magnitude of the retentate pressure for the highest protein concentration for both mAb1 and mA2 is provided in Table 6. From this information, the feed pressure can also be calculated.

TABLE 6
Retentate pressures for mAb1 and mAb2 at the highest protein concentrations achieved using different combinations,
averaged over all the buffer compositions used in this work. Data is presented as Average ± SD.

| | 30-30-30D | | | 50-50-50A | | | 30D-30D-50A | | |
|---|---|---|---|---|---|---|---|---|---|
| | (3 × 30 kDa with D screen) | | | (3 × 50 kDa with A screen) | | | (Hybrid configuration) | | |
| | Max | | Retentate | Max | | Retentate | Max | | Retentate |
| | Concn | ΔP | Pressure | Concn | ΔP | Pressure | Concn | ΔP | Pressure |
| MAb | (g/L) | (psig) | (psig) | (g/L) | (psig) | (psig) | (g/L) | (psig) | (psig) |
| mAb1 | 172 ± 16 | 4.1 ± 0.6 | 17 ± 1 | 243 ± 40 | 4.8 ± 0.1 | 15.1 ± 2.5 | 223 ± 10 | 1.9 ± 0.1 | 10.1 ± 0.3 |
| mAb2 | 56 ± 1 | 5.6 ± 0.1 | 38 ± 3 | 86 ± 0 | 1.8 ± 0.1 | 11.7 ± 0.1 | 191 ± 11 | 1.1 ± 0.2 | 11.5 ± 0.3 |

### REFERENCES

Arunkumar A, Etzel M R (2013) Fractionation of α-lactalbumin from β-lactoglobulin using positively charged tangential flow ultrafiltration membranes. Sep Purif Technol 105:121-8.
Arunkumar A, Etzel M R (2014) Fractionation of α-lactalbumin and β-lactoglobulin from bovine milk serum using staged, positively charged, tangential flow ultrafiltration membranes. J Memb Sci 454:488-95.
Arunkumar A, Singh N, Peck M, Borys M C, Li Z J (2017) Investigation of single-pass tangential flow filtration (SPTFF) as an inline concentration step for cell culture harvest. J Memb Sci 524 (Supplement C):20-32.
Arunkumar A, Singh N, Schutsky E G, Peck M, Swanson R K et al. (2016) Effect of channel-induced shear on biologics during ultrafiltration/diafiltration (UF/DF). J Memb Sci 514 (Supplement C):671-83.
Baek Y, Singh N, Arunkumar A, Borys M, Li Z J et al. (2017) Ultrafiltration behavior of monoclonal antibodies and Fc-fusion proteins: Effects of physical properties. Biotechnol Bioeng 114(9):2057-65.
Bakhshayeshi M, Teella A, Zhou H, Olsen C, Yuan W, Zydney A L (2012) Development of an optimized dextran retention test for large pore size hollow fiber ultrafiltration membranes. J Memb Sci 421-422:32-8.
Binabaji E, Ma J, Rao S, Zydney A L (2016) Ultrafiltration of highly concentrated antibody solutions: Experiments and modeling for the effects of module and buffer conditions. Biotech Prog 32(3):692-701.
Bolton G R, Apostolidis A J (2017) Mechanistic modeling of the loss of protein sieving due to internal and external fouling of microfilters. Biotech Prog 33(5):1323-33.
Brinkmann A, Elouafiq S, Pieracci J, Westoby M (2018) Leveraging single-pass tangential flow filtration to enable decoupling of upstream and downstream monoclonal antibody processing. Biotech Prog 34(2):405-11.
Burns D B, Zydney A L (2001) Contributions to electrostatic interactions on protein transport in membrane systems. AIChE Journal 47(5):1101-14.
Dizon-Maspat J, Bourret J, D'Agostini A, Li F (2012) Single pass tangential flow filtration to debottleneck downstream processing for therapeutic antibody production. Biotechnol Bioeng 109(4):962-70.
Hammerschmidt N, Tscheliessnig A, Sommer R, Helk B, Jungbauer A (2014) Economics of recombinant antibody production processes at various scales: Industry-standard compared to continuous precipitation. Biotech J 9(6):766-75.
Konstantinov K B, Cooney C L (2015) White paper on continuous bioprocessing. May 20-21, 2014 Continuous Manufacturing Symposium. J Pharm Sci 104(3):813-20.
Kwon B, Molek J, Zydney A L (2008) Ultrafiltration of PEGylated proteins: Fouling and concentration polarization effects. J Memb Sci 319(1):206-13.
Lebreton B, Brown A, van Reis R (2008) Application of high-performance tangential flow filtration (HPTFF) to the purification of a human pharmaceutical antibody fragment expressed in Escherichia coli. Biotechnol Bioeng 100(5):964-74.
Manzano I, Zydney A L (2017) Quantitative study of RNA transmission through ultrafiltration membranes. J Memb Sci 544 (Supplement C):272-7.
Nambiar A M K, Li Y, Zydney A L (2018) Countercurrent staged diafiltration for formulation of high value proteins. Biotechnol Bioeng 115(1):139-44.
Pall Corporation Selection Guide: Separation Products for Centrifugal and Tangential Flow Filtration, available at https://laboratory.pall.com/content/dam/pall/laboratory/literature-library/non-gated/id-7046.pdf (last accessed Jun. 5, 2019).
Proteins Used as Molecular-Weight Standards—Proteins and Proteomics, available at http://www.proteinsandproteomics.org/content/free/tables_1/table10.pdf (last accessed Jun. 5, 2019).
Ruanjaikaen K, Zydney A L (2011) Purification of singly PEGylated α-lactalbumin using charged ultrafiltration membranes. Biotechnol Bioeng 108(4):822-9.
Teske C A, Lebreton B, Reis R V (2010) Inline ultrafiltration. Biotech Prog 26(4):1068-72.
Thiess H, Leuthold M, Grummert U, Strube J (2017) Module design for ultrafiltration in biotechnology: Hydraulic analysis and statistical modeling. J Memb Sci 540 (Supplement C):440-53.
Warikoo V, Godawat R, Brower K, Jain S, Cummings D et al. (2012) Integrated continuous production of recombinant therapeutic proteins. Biotechnol Bioeng 109(12):3018-29.
Zydney A L (2015) Perspectives on integrated continuous bioprocessing—opportunities and challenges. Curr Opin Chem Eng 10 (Supplement C):8-13.
Zydney A L (2016) Continuous downstream processing for high value biological products: A review. Biotechnol Bioeng 113(3):465-75.

1. A method for concentrating a solution of a macromolecule that is retained on at least two semi-permeable membranes that have different molecular weight cutoffs (MWCOs), the method comprising passing the solution through a hybrid configuration of said semi-permeable membranes staged in series in a single pass tangential flow filtration (SPTFF) apparatus, wherein the last membrane in the series has a larger MWCO than the preceding membrane or membranes.

2. The method of claim 1, wherein the biological macromolecule is a biological macromolecule.

3. The method of claim 2, wherein the biological macromolecule is chosen from a protein, nucleic acid, DNA, RNA, virus particle, ribonucleoprotein, carbohydrate, glycoprotein, lipid, triglyceride, phospholipid, lipoprotein, and a fragment or portion of any of said biological macromolecules.

4. The method of claim 3, wherein the protein is chosen from a polypeptide, a multimeric protein, an antibody, an antigen-binding portion of an Ab, an antibody-drug conjugate, an immunoconjugate, an Fc portion of an Ab, a Fc fusion protein, a deoxyribonucleoprotein, a ribonucleoprotein (RNP), a small nuclear RNP (snRNP), a RNA virus, a glycoprotein, a lipoprotein, a PEGylated protein, and a fragment or portion of any of said proteins.

5. The method of claim 3, wherein the nucleic is chosen from chromosomal DNA, genomic DNA, cDNA, viral DNA, plasmid DNA, viral vector DNA, vaccine DNA, deoxyribonucleotides, RNA, and ribonucleotides.

6. The method of claim 2, wherein the biological macromolecule has a molecular weight of about 10 to about 20, about 20 to about 40, about 40 to about 60, about 60 to about 90, about 90 to about 120, about 120 to about 160, or greater than about 160 kDa.

7. The method of claim 1, wherein three semi-permeable membranes are used in the SPTFF apparatus.

8. The method of claim 1, wherein the biological macromolecule has a molecular weight of about 90 to about 180 kDa and the membranes are staged in a 20-30, a 20-40, a 25-40, a 25-50, a 30-50, a 20-20-30, a 25-25-40, a 30-30-50, a 20-30-40-50, a 20-20-20-40, a 25-25-25-40, or a 30-30-30- 50 kDa hybrid configuration.

9. The method of claim 8, wherein the biological macromolecule is an antibody and the membranes are staged in a 30-30-50 kDa hybrid configuration.

10. The method of claim 1, wherein the biological macromolecule has a molecular weight of about 30 to about 90 kDa and the membranes are staged in a 5-10, a 5-5-10, a 8-12, a 8-8-12, a 10-15, a 10-10-15, a 12-15, a 12-12-15, a 15-20, a 15-15-20, a 20-30, a 20-20-30, a 15-15-15-20, or a 20-20-20-30 kDa hybrid configuration.

11. The method of claim 1, wherein the biological macromolecule has a molecular weight of about 10 to about 30 kDa and the membranes are staged in a a 3-5, 3-3-5, a 5-8, a 5-5-8, a 5-5-10, a 8-8-10, a 3-5-8-10, a 5-5-5-10, or a 8-8-8-10 kDa hybrid configuration.

12. The method of claim 4 for concentrating an antibody solution, wherein the method achieves a concentration of about 150 to about 200 mg/mL, or a concentration of greater than about 200 mg/mL.

13. The method of claim 4 for concentrating an antibody solution, wherein the method achieves a concentration about or at least about 5, 10, 12, 15, 20, 30, 50, 60, 70, 75, 90, 100, 150 or greater than 150-fold higher than the concentration of the starting solution.

14. The method of claim 4 for concentrating an antibody solution, wherein the hybrid configuration allowed operation at a flow rate at about or at least about 2-fold higher, or about or at least about 4-fold higher, than the maximum flow rate achieved using membranes in a non-hybrid configuration.

15. The method of claim 4 for concentrating an antibody solution, wherein the hybrid 30-30-50 kDa configuration allowed operation at a flow rate about 2-fold higher or at least about 2-fold higher than the maximum flow rate achieved using membranes in a 30-30-30 kDa or 50-50-50 kDa configuration.

16. The method of claim 4, wherein the hybrid 30-30-50 kDa configuration allowed operation at a flow rate about 4-fold higher or at least 4-fold higher than the maximum flow rate achieved using membranes in a 30-30-30 kDa or 50-50-50 kDa configuration.
